(19) United States
(12) Patent Application Publication
(10) Pub. No.: US 2026/0279514 A1
(43) Pub. Date: Sep. 17, 2026

(54) COMPUTER-BASED METHOD TO DETERMINE OPTIMAL SUNSCREEN FILTER COMPOSITION

(71) Applicant: BASF SE, Ludwigshafen am Rhein (DE)

(72) Inventors: David Simon WALZ, Ludwigshafen Am Rhein (DE); Lukas NIEMEIER, Ludwigshafen Am Rhein (DE); Bernd HERZOG, Grenzach-Wyhlen (DE); Myriam SOHN, Grenzach-Wyhlen (DE); Christian CREMER, Grenzach-Wyhlen (DE); David HAJNAL, Ludwigshafen Am Rhein (DE)

(21) Appl. No.: 18/872,033

(22) PCT Filed: Jun. 9, 2023
(86) PCT No.: PCT/EP2023/065532

§ 371 Date: Dec. 5, 2024

(30) Foreign Application Priority Data
Jun. 10, 2022 (EP) 22178368.1

(51) Int. Cl. G16C 60/00; G06F 30/20; G06F 111/06; G16C 20/80

(52) U.S. Cl. CPC G16C 60/00; G06F 30/20; G16C 20/80; G06F 2111/06

(57) ABSTRACT

The present invention relates to sunscreen filter composition development. In order to efficiently determine a sunscreen filter composition, a computer-implemented method (200) is provided for determining a composition comprising one or more UV-filter substances, the method comprising the steps of: a) providing (210), via an input unit, input data that comprises: a sunscreen performance target profile comprising a minimum product requirement of the composition; and a set of optimization objective parameters that comprises two or more optimization objective parameters; b) performing (220), by a processing unit, a multi-objective optimizing process on a computational model to determine a set of Pareto-efficient solutions, wherein each solution is a composition of substances to form the composition such that the composition meets the minimum product requirement; process and each solution is Pareto-efficient with respect to the set of optimization objective parameters; and c) providing (230), via an output unit, at least one of the determined composition of substances, which is preferably usable for guiding the production of a sunscreen product.

### FIELD OF THE INVENTION

The present invention relates to a computer-implemented method and an apparatus for determining a composition comprising one or more UV-filter substances, to a method and an apparatus for manufacturing a sunscreen product, to a method and an apparatus for validating manufacture of a sunscreen product, and to a computer program element.

### BACKGROUND OF THE INVENTION

Sunscreen filter compositions are prepared from a palette of different UV-filter substances which are known for their different UV absorbing characters. When creating a sunscreen filter composition the designer selects substances and combines them and their relative proportions to achieve a target sun protection performance.

In current formulation design of a sunscreen product, the designer often tries to find an optimal solution in the multi-dimensional objective space by an empirical iterative change of the formulation parameters in the design space. Usually, this procedure is continued until either a solution is found which fulfils certain requirements, or some deadline is reached where a solution has to be delivered. For example, EP 3 889 963 A1 describes a computer-based method for determining a sunscreen composition. A user is requested to select the optimization objective. This allows for a user-defined interactive process, where the objective may be selected according to the general needs of the user as well as according to the properties of the sunscreen that shall be optimized. In particular, the objective may be changed during the process in order to iteratively improve the sunscreen composition.

This iterative procedure may lead to good results; however, no guarantee on optimality can be given. Furthermore, the empirical optimization only covers restricted areas in both the design and objective space, so that only limited information on the trade-offs between the different objectives is available and the decision cannot be based on an overview of the full solution space. This limitation may lead to overlooking interesting solutions.

### SUMMARY OF THE INVENTION

There may be a need to efficiently determine a composition comprising one or more UV filter substance.

The object of the present invention is solved by the subject-matter of the independent claims, wherein further embodiments are incorporated in the dependent claims. It should be noted that the following described aspects of the invention apply also for the computer-implemented method and the apparatus for determining a composition comprising one or more UV-filter substances, the method and the apparatus for manufacturing a sunscreen product, the method and the apparatus for validating manufacture of a sunscreen product, and the computer program element.

According to a first aspect of the present invention, there is provided a computer-implemented method for determining a composition comprising one or more UV-filter substances, the method comprising the steps of:
a) providing, via an input unit, input data that comprises:
   a sunscreen performance target profile comprising a minimum product requirement of the composition; and
   a set of optimization objective parameters that comprises two or more optimization objective parameters;
b) performing, by a processing unit, a multi-objective optimizing process on a computational model to determine a set of Pareto-efficient solutions, wherein
   each solution is a composition of substances to form the composition such that the composition meets the minimum product requirement; and
   each solution is Pareto-efficient with respect to the set of optimization objective parameters; and
c) providing, via an output unit, information about at least one of the determined composition of substances, which is preferably usable for guiding the production of a sunscreen product.

The computer-implemented method and apparatus as described herein provide reliable means for multicriteria optimization of a composition comprising one or more UV-filter substances.

Instead of finding an optimal solution in the multi-dimensional objective space by an empirical iterative change of the formulation parameters in the design space, the proposed computer-implemented method and apparatus enable searching for a desired optimal compromise of two or more properties in a simultaneous approach. Thus, more information on trade-offs between the different objectives is available and the decision can be based on this knowledge. The desired optimal solutions are a subset of the feasible solutions.

This may help the users (e.g., a business) to identify and produce suitable product. The number of lab experiments would be reduced to an absolute minimum. This would speed up formulation development and production.

In addition, the generated composition of substances may represent a sunscreen product with a desired performance characteristic. In this way, various substances including UV-filter substances may be checked objectively to validate the user requirements of the performance characteristics, to validate composition before production/delivery, and to tailor sunscreen products to the needs of a user. Thus, the evaluation does not require in vitrol in vivo experimentation.

Furthermore, the computer-implemented method and apparatus as described herein may be useful for exchanging substances in a sunscreen product, which are blocked due to competitive intellectual property rights, regulatory issues in different countries, or lack of resources.

The computer-implemented method will be described hereinafter and in particular with respect to the example shown in FIG. 3.

According to an embodiment of the present invention, the input data further includes one or more technical requirements to restrict the Pareto-efficient solutions to fulfill the one or more technical requirements.

For example, if one or more solvents are used, the one or more UV filters may be dissolved in the one or more solvents, and all given concentrations (i.e., filters & solvents) may comply with solubility data. To ensure this a solubility model may be applied using the solubility data of each filter in each solvent.

According to an embodiment of the present invention, the one or more technical requirements comprises one or more of:
a solubility model comprising solubility data of each solid filters in one or more solvents;
an assumed limit of individual concentrations of each solvent;
an assumed limit of individual concentrations of each filter;
an assumed limit for a total filter concentration; and
an assumed limit of a total solvent concentration.

For example, in the case of a linear solubility model it is assumed that i) for a given filter, the total solubility within a mixture of solvents is given by the fraction-average of the solubility of that filter in the respective solvent and ii) that the solubilities of the individual filters within solvent do not interfere, meaning that i.) is applied for all filters independently.

According to an embodiment of the present invention, each composition of substances comprises at least one of the following:
one or more UV-filter substances;
one or more UV-filter substances and one or more solvents
one or more UV-filter substances in a formulation type; and
one or more UV-filter substances and one or more solvents in a formulation type.

In an embodiment, UV-filter substance may comprise a specific compound that impedes the passage of ultraviolet light. In other words, the UV-filter substance may include soluble or insoluble, organic or inorganic agents that protect the skin from sun damages due to UVB and/or UVA irradiation such as erythema, skin cancers, etc. by absorbing or blocking ultraviolet radiation. The soluble UV-filter agents work by absorbing UV rays. They can be soluble or miscible either in an hydrophilic medium or in a lipophilic medium. The hydrophilic filters are added in the hydrophilic part of a formulation. The lipophilic soluble UV-filters can be supplied in a liquid form and can then be added directly in the lipophilic phase of the emulsion vehicle. Some are supplied as solid UV Filter such as Ethylhexyl Triazone, Diethylamino Hydroxybenzoyl Hexyl Benzoate, Bis-Ethylhexyloxyphenol Methoxyphenyl Triazine, Butyl Methoxydibenzoylmethane, Diethylhexyl Butamido Triazone and need to be dissolved in the lipophilic phase of the emulsion vehicle with solvents. Solvents are here cosmetic oils. The insoluble or particulate UV-filter agents work by absorbing and additionally reflecting and/or scattering UV rays such as Tris-Biphenyl Triazine, Methylene Bis-Benzotriazolyl Tetramethylbutylphenol, Phenylene Bis-Diphenyltriazine, Bis-(Diethylamino Hydroxybenzoyl Benzoyl) piperazine, titanium dioxide, and zinc oxide. The organic particulate filters are generally added in the hydrophilic part of the emulsion (either in the water phase before emulsification or in the external hydrophilic phase of an oil-in-water emulsion) Titanium dioxide and zinc oxide can be added to the lipophilic or hydrophilic part depending on the presence and the type of the coating. Different jurisdictions may allow different UV filters. The UV-filters allowed in cosmetic products in the European Union are listed in the Annex VI of the Regulation “EC” No. 1223/2009 of the European Parliament and of the Council. In the present disclosure, the term “UV filter” is also referred to as “filter”.

The formulation vehicle, which is also referred to as formulation type, of sunscreens should enable a homogeneous dispersion of the active UV filter in the vehicle and on the skin. The formulation types may impact the sun protection performance. Therefore, different formulations with the same UV-filter composition may exhibit different sun protection performances.

Towards this end, the computer-implemented method and the apparatus as described herein may consider the formulation type (e.g., fluid emulsion, cream emulsion, etc.) in addition to the properties of UV filters. The choice of the formulation type is dependent on individual requirements and preferences. Having the possibility of selecting the formulation type, the desired solution may be tailored to the user's needs. The desired solution may also reduce the difference between the target sun protection performance index and the measured sun protection performance in vivo, thereby reducing extensive in vivo testing's at the user level.

The formulation type may comprise a formulation with water or a formulation without water. Examples of the formulation type may include, but are not limited to, oil-in-water fluid emulsion, oil-in-water cream emulsion, water-in-oil emulsion, oil-in-water in oil emulsion, water in oil in water emulsion, water in silicone emulsion, silicone-in-water emulsion, polymeric gel cream, lipophilic monophase oil, lipophilic monophase gel, lipophilic monophase stick, lipophilic-alcoholic mixture, hydrophilic monophase fluid, hydrophilic monophase gel, and powder.

According to an embodiment of the present invention, the method further comprises the step of providing a graphical user interface (GUI) for Pareto Front visualization, wherein the GUI provides an interactive user interface allowing a user to navigate on the set of determined Pareto-efficient solutions through an adjustment of the set of optimization objective parameters and/or an adjustment of a formulation of the at least one of the determined composition of substances.

An exemplary interactive user interface will be described in detail with respect to the example shown in FIG. 4A and FIG. 4B.

According to an embodiment of the present invention, an interpolation between points in the set of determined Pareto-efficient solutions is performed, and the GUI provides the interactive user interface allowing the user to navigate on the interpolated Pareto-efficient solutions.

This will be explained in detail hereinafter and in particular with respect to the example shown in FIG. 5.

According to an embodiment of the present invention, the minimum product requirement of the composition comprises one or more of: Sun Protection Factor (SPF), UVA Protection Factor (UVA-PF), critical wavelength, ratio of UVA to UVB protection, ratio of UVA1 to UV protection, blue light protection, Radical protection factor, environmental impact factor, limit concentration of a substance, and formulation type.

The implementation of the one or more minimum product requirement may be tackled by translating the one or more minimum product requirement in at least one corresponding mathematical optimization constraint.

In an embodiment, the sun protection factor (SPF) may indicate the protection of the skin against the occurrence of an erythema. The factor indicates how much longer the protected skin may be exposed to the sun without getting a sunburn in comparison to untreated skin. For example, if a sunscreen composition with an SPF of 15 is evenly applied to the skin of a person usually getting a sunburn after 10 minutes in the sun, the sunscreen allows the skilled person to stay in the sun 15 times longer. In other words, SPF 15 means that 1/15 of the burning UV radiation will reach the skin, assuming sunscreen is applied evenly at a thick dosage of 2 milligrams per square centimetre (mg/cm2). It can be evaluated in vivo (according to ISO24444), or can be calculated with the BASF sunscreen simulator (https://sunscreensimulator.basf.com/Sunscreen_Simulator/login), or in vitro using transmittance measurement through a thin film of sunscreen sample spread on roughened substrate plate which is transparent to UV.

A suitable substrate is for example PMMA plates. In vitro transmission measurement may be performed from 290-400 nm with 1 nm steps with the Labsphere UV Transmittance Analyzer UV 2000S. The UV transmission spectrum is then acquired and the SPF in vitro values are calculated according to equation below.

$$SPF = \frac{{\sum }_{2 \; 90 nm}^{400 nm} {s}_{er} ( λ ) \cdot S ( λ )}{{\sum }_{2 \; 90 nm}^{400 nm} {s}_{er} ( λ ) \cdot S ( λ ) \cdot T ( λ )}$$

where, ser(λ) is the erythema action spectrum and S(λ) is the spectral irradiance received from the UV source and T(λ) is the in vitro measured light transmittance.

In an embodiment, UVA Protection Factor (UVA-PF) may indicate the protection of the skin against UVA rays and can be measured according to ISO24442 or ISO24443 or can be calculated with the BASF sunscreen simulator (https://sunscreensimulator.basf.com/Sunscreen_Simulator/login). It is recommended by the European Commission that all sunscreens should have an UV-A protection factor, which is at least one third of the labelled sun protection factor (SPF), e.g., if the sunscreen composition has an SPF of 30 the UVA protection factor has to be at least 10. In Asia a PA classification is recommended to characterize the UVA protection of a sunscreen according to the UVA-PF value. As an example, a PA+++ classification indicates a UVA-PF value between 8 and 16 and PA++++ when the UVA-PF value is higher than 16.

In an embodiment, critical wavelength may correspond to the wavelength below which 90% of the area under the absorbance curve between 290 and 400 nm is covered. It is an in vitro test used to determine the UV absorbance in the UVA range. According to the US requirements, a critical wavelength of at least 370 nm is required for achieving broad spectrum protection. In the proposed order (OTC000008) published Sep. 24, 2021, the FDA proposed to add an additional requirement to pass the broad-spectrum test, the product shall meet a UVA1/UV ratio of at least 0.7.

In an embodiment, ratio of UVA1 to UVB protection may correspond to the protection in the UVA1 range (340 nm to 400 nm) compared to the UV range (290 nm to 400 nm),

In an embodiment, ratio of UVA to UVB protection may correspond to the protection in the UVA range (320 nm to 400 nm) compared to the UVB range (290 nm to 320 nm), it can be for example evaluated according to the Boots star rating system method.

In an embodiment, blue light Protection may indicate the protection of the skin against blue light rays (400-500 nm, more preferably, 400 to 450 nm). It can be for example evaluated by transmittance measurements and gives the reduction of the transmittance through a thin film of sunscreen sample spread on roughened substrate plate which is transparent to UV and expressed as a reduction of the transmittance in the blue light range

In an embodiment, radical protection factor may indicate the protection of the skin against the formation of free radicals which formation is wavelength dependent, with two maxima, one in the UVB and one in the UVA range, as described by ZASTROW et al. The missing link--light induced free radical formation in human skin, Skin Pharmacol Physiol, 2009.

In an embodiment, environmental impact factor may indicate the impact on the environment of a specific UV filter composition. This can be evaluated for example with the Ecosun Pass criterium as described in WO2019/207129 A1 which cites that a filter combination with a Ecosun Pass value of at least 200 is required for best environmental compatibility. The environmental impact factor” can be evaluated for example also with the Eco-score factor as described in Kunze et al (New method for connecting sunscreens with consumers via a relative Eco-score, SOFWjournal, 147, 11/21). The objective is to have the lowest environmental impact factor or inversely the highest environmental friendliness.

In one embodiment, the minimum product requirement is a specific SPF value and UVA-PF/SPF ratio of at least ⅓ and critical wavelength of at least 370 nm.

In another embodiment, the minimum product requirement is a specific SPF value and UVA-PF/SPF ratio of at least ⅓ and critical wavelength of at least 370 nm and an Ecosun pass value of at least 200.

In another embodiment, the minimum product requirement is a specific SPF value and UVA1/UV ratio of at least 0.7 and critical wavelength of at least 370 nm.

In a further embodiment, the minimum product requirement is a specific SPF value and UVA-PF of at least 16.

In one embodiment, the minimum product requirement is a specific SPF value and UVA-PF/SPF ratio of at least ⅓, critical wavelength of at least 370 nm and a given maximum total solvent concentration

In one embodiment, the minimum product requirement is a specific SPF value and UVA-PF/SPF ratio of at least ⅓, critical wavelength of at least 370 nm and a given maximum concentration of at least one solvent

In one embodiment, the minimum product requirement is a specific SPF value and UVA-PF/SPF ratio of at least ⅓, critical wavelength of at least 370 nm and a given maximum total filter concentration

In one embodiment, the minimum product requirement is a specific SPF value and UVA-PF/SPF ratio of at least ⅓, critical wavelength of at least 370 nm and a given maximum total filter concentration for an oil in water fluid formulation type

According to another embodiment of the present invention, the input data may further include one or more technical requirements to restrict the Pareto-efficient solutions to fulfill the one or more technical requirements.

In an example, the one or more technical requirements may include one or more of:
a solubility model comprising solubility data of each solid filters in solvents;
an assumed limit of individual concentrations of each solvent;
an assumed limit of individual concentrations of each filter;
an assumed limit for a total filter concentration; and
an assumed limit of a total solvent concentration.

For example, in the case of a linear solubility model it is assumed that i) for a given filter, the total solubility within a mixture of solvents is given by the fraction-average of the solubility of that filter in the respective solvent and ii) that the solubilities of the individual filters within solvent do not interfere, meaning that i.) is applied for all filters independently.

According to an embodiment of the present invention, the set of optimization objective parameters comprises two or more of: minimum of required UV filter concentration, filtering efficiency, number of UV-filters used, minimum concentration of particulate filters, type of UV filter, minimum concentration of a specific UV filter, minimum concentration of a specific solvent, concentration of solvent, type of solvent, minimum concentration of solvent, highest sun protection factor, highest UVA protection factor, highest UVA/UVB ratio, highest UVA1/UV ratio, highest blue light protection, highest environmental friendliness, highest biodegradability, highest naturality, minimized costs of the determined composition of substances, and similarity to a provided composition of filter substances.

Solvent is required to dissolve efficiently the solid UV filters. Examples of solvents used to dissolve solid UV filters include C12-15 Alkyl Benzoate, Dibutyl Adipate, Diisopropyl Sebacate, Dibutyl Sebacate, Dicaprylyl Carbonate, Isopropyl Palmitate, Isopropyl Myristate, lauryl lactate, Caprylyl Capric Triglyceride, Cetearyl Isononanoate, Cocoglyceride, Isononyl Isononanoate, Propylene Glycol Dicaprylate/Dicaprate Butylene Glycol Dicaprylate/Dicaprate, etc.

The type of filter may refer to its solubility for example if the filter is intended to be added in the hydrophilic or lipophilic phase of the formulation, or might refer to its form for example if the filter is particulate or not particulate or to its biodegradation profile for example if the filter is primary readily biodegradable or moderately biodegradable.

Examples of UV-filters which need to be dissolved are bis-ethylhexyloxyphenol methoxyphenyl triazine, ethylhexyl triazone, Diethylamino Hydroxybenzoyl Hexyl Benzoate, Butyl Methoxydibenzoylmethane, Diethylhexyl Butamido Triazone.

When for example “minimized concentration of solvents” is used as an optimization objective parameter, the Pareto-efficient solutions should ensure that all solid UV filters are dissolved in the solvents. For example, a linear solubility model can be used which assumes that i) for a given filter, the total solubility within a mixture of solvents is given by the fraction-average of the solubility of that filter in the respective solvent and ii) that the solubilities of the individual filters within solvent do not interfere, meaning that i.) is applied for all filters independently. In other words, if solvents are used as optimization objectives, the solid UV filters need to be dissolved in the amount of solvents given either as “minimized optimization objectives” or as “Pareto-efficient solutions”.

According to a second aspect of the present invention, there is provided a method for manufacturing a sunscreen product, the method comprising the steps of:
providing a target performance characteristic of a desired sunscreen product;
determining, based on the target performance characteristic, a composition comprising one or more UV-filter substances according to the method of any one of the preceding claims; and
manufacturing a sunscreen product using the composition comprising the one or more UV-filter substances.

This will be explained in detail hereinafter and in particular with respect to the example shown in FIG. 6.

According to an embodiment of the present invention, the method further comprises:
providing a measured performance characteristic of the manufactured sunscreen product; and
comparing the measured performance characteristic of the manufactured sunscreen product with the target performance characteristic of the desired sunscreen product to determine if the manufactured sunscreen product fulfils predetermined performance quality criteria.

According to a third aspect of the present invention, there is provided a method for validating manufacture of a sunscreen product, the method comprising the steps of:
providing an existing performance characteristic for a sunscreen product that has been produced from one or more substances;
generating a composition based on the existing performance characteristic according to the method of the first aspect and any associated example, wherein the generated composition and the existing sunscreen product comprise at least one different substance;
producing the generated composition; and
comparing a measured performance characteristic of produced composition and the existing performance characteristic of the sunscreen product to validate the at least one substance.

This will be explained in detail hereinafter and in particular with respect to the example shown in FIG. 7.

According to a fourth aspect of the present invention, there is provided an apparatus for determining a composition comprising one or more of UV-filter substances, the apparatus comprising one or more processing units configured to determine a composition comprising one or more UV-filter substances to form the sunscreen composition, wherein the one or more processing units include instructions, which when executed on the one or more processing units execute the method steps of the method according to the first aspect and any associated example.

This will be explained in detail hereinafter and in particular with respect to the examples shown in FIGS. 1 and 2.

According to a fifth aspect of the present invention, there is provided an apparatus for manufacturing a sunscreen product. The apparatus comprises a controller module, and a manufacturing device. The controller module is configured to control the manufacturing device to manufacture the sunscreen product.

This will be explained in detail hereinafter and in particular with respect to the example shown in FIG. 8.

According to a sixth aspect of the present invention, there is provided an apparatus for validating manufacture of a sunscreen product, the apparatus comprising one or more processing unit(s) configured to validate production of a sunscreen product, wherein the processing unit(s) include instructions, which when executed on the one or more processing unit(s) preform the method according to the third aspect and any associated example.

This will be explained in detail hereinafter and in particular with respect to the example shown in FIG. 9.

According to a further aspect of the present invention, there is provided a computer program element comprising instructions, which when executed by a processing unit, cause the processing unit to carry out the steps of the method according to the first aspect and any associated example.

In an embodiment, the sunscreen product may comprise a finished formulation for protection of human skin from ultraviolet radiation. Sunscreens contain one or more ultraviolet (UV) filter substances that may include organic and/or inorganic UV filters. In addition, a sunscreen product may contain many other substances, such as solvent, emulsifiers, thickeners, waxes, preservatives or stabilizers, fragrances, and colouring compounds.

In an embodiment, input unit may comprise any item or element forming a boundary configured for transferring information. In particular, the input unit may be configured for transferring information onto a computational device, e.g. onto a computer, such as to receive information. The input unit preferably is a separate unit configured for receiving or transferring information onto a computational device, e.g. one or more of: an interface, specifically a web interface and/or a data interface; a keyboard; a terminal; a touchscreen, or any other input device deemed appropriate by the skilled person. More preferably, the input unit comprises or is a data interface configured for transferring or exchanging information as specified herein below.

In an embodiment, output unit may comprise any item or element forming a boundary configured for transferring information. In particular, the output unit may be configured for transferring information from a computational device, e.g. a computer, such as to send or output information, e.g. onto another device, e.g. a control unit, that controls and/or monitor the production process of the produced composition. The output unit preferably is a separate unit configured for outputting or transferring information from a computational device, e.g. one or more of: an interface, specifically a web interface and/or a data interface; a screen, a printer, or a touchscreen, or any other output device deemed appropriate by the skilled person. More preferably, the output unit comprises or is a data interface configured for transferring or exchanging information as specified herein below.

Preferably, the input unit and the output unit are configured as at least one or at least two separate data interface(s); i.e. preferably, provide a data transfer connection, e.g. a wireless transfer, an internet transfer, Bluetooth, NFC, inductive coupling or the like. As an example, the data transfer connection may be or may comprise at least one port comprising one or more of a network or internet port, a USB-port and a disk drive. The input unit and/or the output unit may also be may be at least one web interface.

In an embodiment, processing unit may refer to an arbitrary logic circuitry configured for performing operations of a computer or system, and/or, generally, to a device or unit thereof which is configured for performing calculations or logic operations. The processing unit may comprise at least one processor. In particular, the processing unit may be configured for processing basic instructions that drive the computer or system. As an example, the processing unit may comprise at least one arithmetic logic unit (ALU), at least one floating-point unit (FPU), such as a math coprocessor or a numeric coprocessor, a plurality of registers and a memory, such as a cache memory. In particular, the processing unit may be a multi-core processor. The processing unit may comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like. The processing unit may be configured for pre-processing the input data. The pre-processing may comprise at least one filtering process for input data fulfilling at least one quality criterion. For example, the input data may be filtered to remove missing variables. Preferably, input data may be compared to at least one pre-defined threshold value, e.g. a threshold temperature, to determine whether method step (ii) is required to be performed at all. Preferably, the processing unit is configured to perform a multicriterial optimization, preferably calculation, of an optimization signal consisting of a list of input parameters leading according to the underlying model prediction to optimized target application profiles. Preferentially, the multicriterial optimization is a Pareto optimization and this list consists of Pareto optimal solutions. Furthermore the optimization signal may contain a complete or approximate representation of the Pareto frontier based on the underlying model function.

In an embodiment, computational model may refer to the model used for simulations of sunscreen performance which is based on the calculation of UV-transmissions of sunscreen films. The link to the sun protection factor is given by the fact, that the inverse of transmittance at a specific wavelength is the factor by which the respective radiation is attenuated and has the meaning of a monochromatic protection factor. Weighting of this monochromatic protection factor by the erythemal action spectrum and the spectrum of the UV-light source in the relevant UV-range of 290 to 400 nm yields the sun protection factor. While data for erythemal action spectrum and UV-light source are available from literature, the transmittance has to be determined for each sunscreen composition individually. Calculations of UV-transmittance require quantitative UV-spectra of the UV-filters, for instance in terms of molar decadic extinction coefficients. From such spectral data the effective UV-absorbance of the filter composition can be obtained. However, in order to get realistic UV-transmittances, the irregular structure of the sunscreen film which forms on the skin must be taken into consideration. In addition, since some UV-absorbers are not photostable, information concerning the photokinetics of the UV filters is also necessary. An explanation/description of the computational model is described in Bernd Herzog and Uli Osterwalder. Simulation of sunscreen performance. Pure Appl. Chem. 2015; 87(9-10): 937-951

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 illustrates a block diagram of an exemplary apparatus for determining a composition comprising one or more UV-filter substances.

FIG. 2 illustrates an exemplary system for determining a composition comprising one or more UV-filter substances.

FIG. 3 illustrates a flowchart describing a computer-implemented method for determining a composition comprising one or more UV-filter substances.

FIG. 4A and FIG. 4B illustrate exemplary implementations of the user interface in form of interactively movable sliders.

FIG. 5 illustrates a navigation on the Pareto front of two minimization objectives A and B

FIG. 6 illustrates a flow chart describing a method for providing manufacturing a sunscreen product.

FIG. 7 shows an example of a flowchart describing a method for validating manufacture of a sunscreen product.

FIG. 8 shows an example of a production line for manufacturing a sunscreen product with a monitoring apparatus.

FIG. 9 shows another example of a production line for manufacturing sunscreen product with a validation apparatus.

### DETAILED DESCRIPTION OF EMBODIMENTS

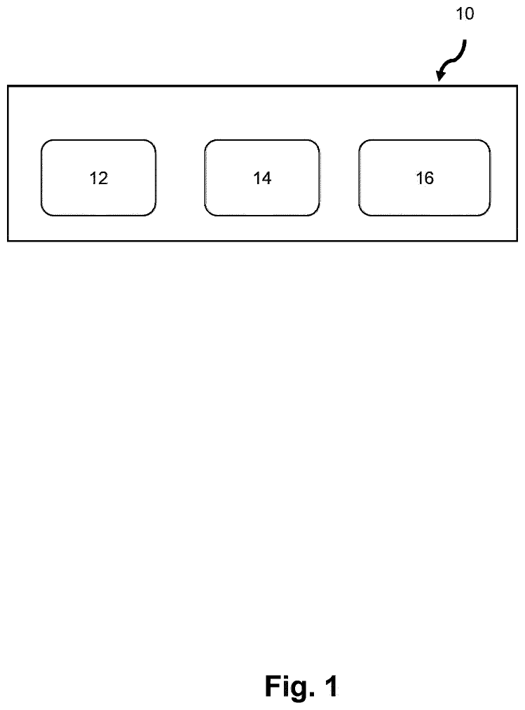

FIG. 1 illustrates a block diagram of an exemplary apparatus 10 for determining a composition comprising one or more UV-filter substances. The apparatus 10 includes an input unit 12, a processing unit 14, and an output unit 16.

In general, the apparatus 10 may comprise various physical and/or logical components for communicating and manipulating information, which may be implemented as hardware components (e.g. computing devices, processors, logic devices), executable computer program instructions (e.g. firmware, software) to be executed by various hardware components, or any combination thereof, as desired for a given set of design parameters or performance constraints. Although FIG. 1 may show a limited number of components by way of example, it can be appreciated that a greater or a fewer number of components may be employed for a given implementation.

In some implementations, the apparatus 10 may be embodied as, or in, a device or apparatus, such as a server, workstation, or mobile device. The apparatus 10 may comprise one or more microprocessors or computer processors, which execute appropriate software. The processing unit 14 of the apparatus 10 may be embodied by one or more of these processors. The software may have been downloaded and/or stored in a corresponding memory, e.g. a volatile memory such as RAM or a non-volatile memory such as flash. The software may comprise instructions configuring the one or more processors to perform the functions described herein.

It is noted that the apparatus 10 may be implemented with or without employing a processor, and also may be implemented as a combination of dedicated hardware to perform some functions and a processor (e.g. one or more programmed microprocessors and associated circuitry) to perform other functions. For example, the functional units of the apparatus 10, e.g. the input unit 12, the one or more processing units 14, and the output unit 16 may be implemented in the device or apparatus in the form of programmable logic, e.g. as a Field-Programmable Gate Array (FPGA). In general, each functional unit of the apparatus may be implemented in the form of a circuit.

In some implementations, the apparatus 10 may also be implemented in a distributed manner. For example, some or all units of the apparatus 10 may be arranged as separate modules in a distributed architecture and connected in a suitable communication network, such as a 3rd Generation Partnership Project (3GPP) network, a Long Term Evolution (LTE) network, Internet, LAN (Local Area Network), Wireless LAN (Local Area Network), WAN (Wide Area Network), and the like.

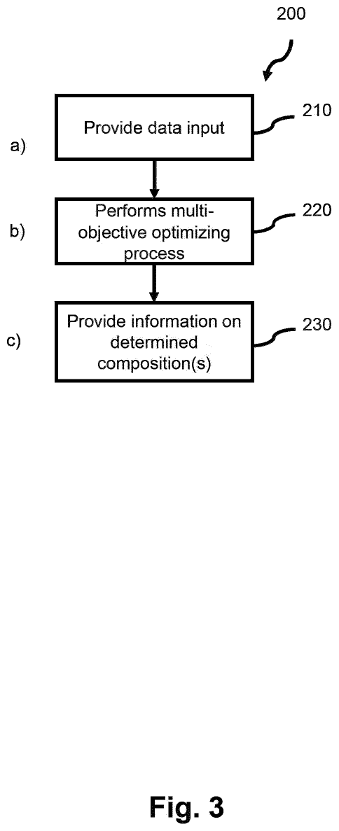

The processing unit(s) 14 may execute instructions to perform the method described herein, which will be explained in detail with respect to the example shown in FIG. 3.

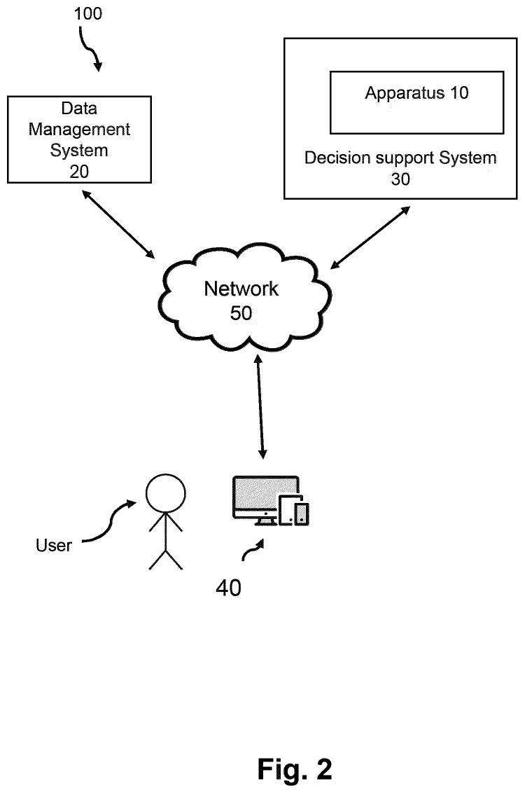

FIG. 2 shows an exemplary system 100 for determining a composition comprising one or more UV-filter substances. The system 100 comprises a data management system 20, a decision support system 30, an electronic communication device 40, and a network 50. In this example, the apparatus 10 is embodied as, or in, the decision support system 30, e.g., residing in the decision support system 30 as a software.

The data management system 20 of the illustrated example may store databases, applications, local files, or any combination thereof. The data management system 20 may comprise a UV-filter substance database that stores information about a plurality of UV-filter substances and properties. For example, the information may include an identifier of the UV-filter substances (e.g., UV-filter substances IDs or a common or scientific name of the UV-filter substances) and related properties. The regulatory limits for some UV-filter substances in some particular regions may also be included in the UV-filter substance database. In some examples, the UV-filter substance database may cover all or most of the common UV-filter substances. In some examples, the UV-filter substance database may be limited to UV-filter substances of a certain provider. Moreover, it is also possible to limit the UV-filter substance database to UV-filter substances allowed in a certain jurisdiction. The UV-filter substance database may be provided by a third party. However, it is also possible that a user can create a tailored UV-filter substance database by scanning the labels of a UV-filter substance product he/she intends to use and by acquiring the information about the UV-filter substance product from supplier databases. By means of the latter, it is also possible that a user supplements a UV-filter substance database with information about further UV-filter substances.

The decision support system 30 of the illustrated example may be a server that provides a web service to facilitate management of data. The decision support system 30 may comprise a data extraction module (not shown) configured to identify data in the data management system 20 that is to be extracted, retrieve the data from the data management system 20, and provide the retrieved data to the apparatus 10, which processes the extracted data according to the method as described herein. The processed data and the final outputs of the apparatus 10 may be provided to a user output device (e.g., the electronic communication device 40) and/or in an output database (e.g., in the data management system 20). The user output device may include a computer, a smartphone, a tablet, a smartwatch, a monitor, a data storage device, or any other device, by which a user, including humans and robots, can receive data from the decision support system, such as the electronic communication device 40. The output database may comprise any organized collection of data, which can be stored and accessed electronically from a computer system, and which can receive data, which is outputted or transferred from the decision support system 30.

The electronic communication device 40 of the illustrated example may be a desktop, a notebook, a laptop, a mobile phone, a smart phone, and/or a PDA. The electronic communication device 40 may comprises an application configured to interface with the web service provided by the decision support system 30. The application may be a software application that enables a user to manipulate data extracted from the data management system 20 by the decision support system 30 and to select and specify actions to be performed on the individual data. For example, the application may be a desktop application, a mobile application, or a web-based application. The application may comprise a user interface, such as an interactive interface including, but not limited to, a GUI, a character user interface, and a touch screen interface. Via the software application, the user may access the decision support system 30 using e.g., Username and Password Authentication to obtain data from the data management system 20 and to determine a composition comprising one or more UV-filter substances.

The network 50 of the illustrated example communicatively couples the data management system 20, the decision support system 30, and the electronic communication device 40. In some examples, the network 50 may be the internet. Alternatively, the network 50 may be any other type and number of networks. For example, the network 50 may be implemented by several local area networks connected to a wide area network. For example, the data management system 20 may be associated with a first local area network, the decision support system 30 may be associated with a second local area network, and the electronic communication device 40 may be associated with a third local area network. The first, second, and third local area networks may be connected to a wide area network. Of course, any other configuration and topology may be utilized to implement the network 50, including any combination of wired network, wireless networks, wide area networks, local area networks, etc.

FIG. 3 illustrates a flowchart describing a computer-implemented method 200 for determining a composition comprising one or more UV-filter substances. In the following, an exemplary order of the steps according to the present disclosure is explained.

The method 200 may be carried out by an apparatus, such apparatus 10 shown in FIG. 1 or FIG. 2. The apparatus may be a computing device or a computing system, regardless of the platform, being suitable for executing program code related to the proposed method. As a further example, the apparatus may be embodied as, or in, a computer system. The apparatus may be embodied as, or in, a remote server that provides a web service to facilitate the determination of a composition comprising one or more UV-filter substances.

In the following, the computer-implemented method 200 of FIG. 3 is described with respect to an exemplary computing environment shown in FIG. 2.

At block 210, via an UI of a software application on the electronic communication device 40, a user can provide data input to the decision support system 30.

The data input comprises a sunscreen performance target profile comprising a minimum product requirement of the composition. The minimum product requirement of the composition may be defined by the user via the UI of the application. For example, the UI of the application may provide a list of properties relating to the sunscreen performance target, such as estimated Sun Protection Factor (SPF), estimated UVA Protection Factor (UVA-PF), critical wavelength, ratio of UVA to UVB protection, ratio of UVA1 to UV protection, blue light protection, Radical protection factor, and environmental impact factor. The user may select one or more of these properties as sunscreen performance target profile. For example, the user may define the following minimum product requirement of the composition: estimated SPF≥50, critical wavelength≥370 nm, and UVA-PF≥17. Additionally, the user may define in the minimum product requirement of the composition containing solid UV filters that the solid UV filters need to be fully dissolved when a minimized concentration of solvent is chosen as an optimization objective.

The data input further comprises a set of optimization objective parameters that comprises two or more optimization objective parameters. The list of optimization objective parameters may be presented on the UI of the software application. For example, the following list of optimization objective parameters may be presented: minimum of required UV filter concentration, minimum concentration of a specific UV filter, minimum concentration of particulate filters, filtering efficiency, number of UV-filters used, type of filters, concentration of solvent, minimum concentration of a specific solvent, type of solvent, minimum concentration of solvent, highest sun protection factor, highest UVA protection factor, highest blue light protection, highest environmental friendliness, highest biodegradability, highest naturality, minimized costs of the determined composition of substances, and similarity to a provided composition of filter substances. The user may select two or more optimization objective parameters from the list of optimization objective parameters.

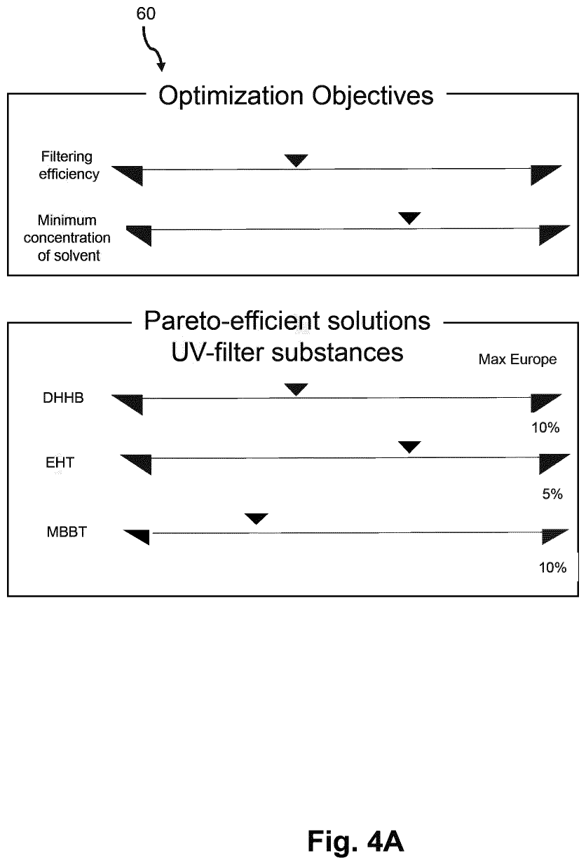

Furthermore, also, in the case that the determination of the optimized concentration of solvent(s) is given by the Pareto optimization as a solution, the optimized concentration of the solvent(s) must ensure that the optimized concentration of the UV filter(s) given by the Pareto optimization as a solution must be dissolved. An example is given in FIG. 4B.

In some examples, the user may select an actual set of UV-filter substances to be considered. For example, a list of UV-filter substances may be displayed and the user may select the desired UV-filter substances. In some examples, the user may select a desired UV-filter database from the data management system 20, such as a UV-filter substance database comprising UV-filter substances of a certain provider, or allowed in a certain jurisdiction, or a user-defined UV-filter substance database. In some examples, the decision support system 30 may extract information about UV-filter substances from a default UV-filter substance database in the data management system 20. In this case, the user is not required to select UV-filter substances. In some examples, the user may have the opportunity to define the minimum and maximum amounts of at least some of the UV-filter substances. In some examples, the data support system may define the maximum amounts of the substances according to the regulatory limits in a UV-filter substance database in the data management system 20.

At block 220, after the user has provided the sunscreen performance target profile and the set of optimization objective parameters to the decision support system 30, the processing unit 12 of the apparatus 10 performs a multi-objective optimizing process on a computational model to determine a set of Pareto-efficient solutions. Each solution is a composition of substances to form the composition such that the composition meets the minimum product requirement. Each solution is Pareto-efficient with respect to the set of optimization objective parameters. Each composition of substances may comprise one or more UV-filter substances, and additionally one or more other ingredients such as emollients.

Pareto optimality refers herein to the concept that a solution is a Pareto improvement if a change to a different solution makes at least one objective better off without making any other objectives worse off. A Pareto improvement is Pareto optimal or Pareto efficient if no further Pareto improvement can be made. With a Pareto frontier, by restricting attention to a set of objectives that are Pareto optimal, the chemist can make trade-offs within such a set, rather than considering the full range of every parameter.

A Pareto frontier is a set of solutions in an N-dimensional objective space that are Pareto optimal in light of a defined method of evaluation of those solutions. For the purposes of forming optimization signals, an N-dimensional Pareto frontier comprises a collection of optimization signals which accommodate the objectives of optimization.

In practice, model based prediction is often carried out by models including a dimension reduction algorithm. Examples are principal component regression or partial least squares models. In these models, so-called latent variables are formed and the target variables, which later serve as optimization objectives are modelled as functions of these latent variables rather than as functions of the original input variables. If the dimension of the space defined by the latent variables is smaller than the dimension of the space defined by the original input parameter for the considered system, then the model exhibits the feature of dimension reduction, and we call the model degenerated. This degeneracy has technical consequences. If a real-world-system is described by a degenerated model with sufficiently high accuracy, then it is possible to systematically change the original input parameters in such a way that none of the target variables of interest are significantly changed.

The space defined by the set of all possible accessible points in input space with this property is referred to as the “invariant subspace”. In the proposed approach, statistical optimality principles on the invariant subspace are combined with applied constrains on the original input parameter space to obtain a set of input parameters for the sunscreen product design. The input parameters may also be referred to as design parameters and are determined in such a way, that the obtained set of input parameters exhibit optimal statistical variability and simultaneously the target variables of interest (i.e. optimization objective parameters, optimization signal) exhibit theoretically zero or in practice technically only minimal variability. The results generated by the proposed approach can be used to cover not only one or just a few lead-recipes, but the entire class of recipes covered by the invariant subspace.

Preferably the GUI allows the user to navigate the interpolated solutions by moving sliders corresponding to the objectives. An exemplary implementation of the user interface is shown in FIG. 4A to 4B.

At block 230, information about at least one of the determined Pareto efficient composition of substances is provided, e.g., to be presented on the UI of the software application on the electronic communication device 40, or to the data management system 20. In some examples, the at least one of the determined composition of substance may be randomly selected by the apparatus 10. In some examples, the at least one of the determined composition of substances may be selected according to a predefined rule. For example, the UV filter combination may be selected and presented according to the filtering efficiency or to the minimum concentration of UV filters. The output information may further comprise an amount (concentration) of each substance. In some examples, the output information may comprise numerous properties characterizing the composition, which may include one or more of the achieved performance, the UVA-PF/SPF ratio, the total concentration of solvents, and the total concentration of UV-filter substances. The information is preferably usable for guiding the production of a sunscreen product.

A sunscreen product may be formulated as fluid or creamy oil-in-water emulsion, etc. On application, the formulation forms a thin film on the skin surface that affords UV protection. Other formulation types may include, but are not limited to, water-in-oil emulsion, oil-in-water in oil emulsion, water in oil in water emulsion, water in silicone emulsion, silicone-in-water emulsion, polymeric gel cream, lipophilic monophase oil, lipophilic monophase gel, lipophilic monophase stick, lipophilic-alcoholic mixture, hydrophilic monophase fluid, hydrophilic monophase gel, and powder. The formulation may be a spray, a cream, a lotion, a mousse (foam), or a powder, and might be packed accordingly in a bottle, a jar, a pump spray, an aerosol with an appropriate applicator. Different formulations with the same UV-filter composition may exhibit different sun protection performances, as the vehicles used in the composition may impact the sun protection performance. In some examples, the above-described computer-implemented method 200 may determine the composition for a specific formulation type (e.g., oil in water fluid emulsion, oil in water cream emulsion, etc.). For example, the user may define or select a desired formulation type (e.g., oil in water fluid emulsion, oil in water cream, emulsion etc.) via the UI of a software application on the electronic communication device 40. The processing unit 12 of the apparatus 10 then performs a multi-objective optimizing process on a computational model to determine a set of Pareto-efficient solutions. Each solution is a composition of substances to form the composition in the desired formulation type, which meets the minimum product requirement. Then, information about at least one of the determined Pareto efficient composition of substances in the desired formulation type is provided, e.g., to be presented on the UI of the software application on the electronic communication device 40, or to the data management system 20. The consideration of the specific formulation type in the determination of the composition may reduce the difference between the target sun protection performance and the measured sun protection performance in vivo, thereby reducing extensive in vivo testing's at the user level.

FIG. 4A to 4B show possible implementations of the user interface 60 in form of interactively movable sliders. In FIG. 4A example, three exemplary U-filter substances and two optimization objectives are considered. The three exemplary UV-filter substances are DHHB (INCI: Diethylamino Hydroxybenzoyl Hexyl Benzoate) (maximum concentration allowed in Europe is 10%), EHT (INCI: Ethylhexyl Triazone) (maximum concentration allowed in Europe is 5%) and MBBT (INCI: Methylene Bis-Benzotriazolyl Tetramethylbutylphenol) (maximum concentration allowed in Europe is 10%). The two exemplary optimization objectives are filtering efficiency and minimum concentration of solvent. By moving one of the objective sliders the other one is automatically adjusted by the system to follow the Pareto frontier. At the same time, the amount of the UV-filter substances leading to the corresponding position on the Pareto Frontier is computed and indicated by an automatic positioning of the input sliders. In this example, the regulatory limits for the three exemplary UV-filter substances in a particular region are also specified.

In the example shown in FIG. 4B, the two optimization objectives are minimum concentration of UV filters and minimum concentration of solvent. The minimum product requirement comprises: SPF of at least 30, UVA-PF of at least 10, critical wavelength of at least 370 nm, and all solid UV filters need to be dissolved. The UV-filter substances are DHHB, EHT, BEMT (INCI: Bis-Ethylhexyloxyphenol Methoxyphenyl Triazine) and DBT (INCI: Diethylhexyl Butamido Triazone). All are solid UV filters. By moving one of the objective sliders the other one is automatically adjusted by the system to follow the Pareto frontier. At the same time, the amount of the UV-filter substances leading to the corresponding position on the Pareto Frontier is computed and indicated by an automatic positioning of the input sliders. The right-angle triangles on each side of the sliders enable to restrict the range of the corresponding optimization objectives or determined substance concentration. In this example, also the optimized concentration of four emollients are shown including Cetiol AB (INCI: C12-15 alkyl benzoate); Cetiol B (INCI: dibutyl adipate); Myritol 331 (INCI: Cocoglycerides); Cetiol CC (INCI: Dicaprylyl Carbonate). Each Pareto-efficient solution ensure that all solid UV filters (DHHB, BEMT, DBT; EHT) are dissolved in the determined Pareto-efficient concentration of solvents.

Table 1 shows examples of solutions of Pareto frontier optimization for of a sunscreen with SPF 50 and UVA-PF/SPF protection of at least ⅓. In the examples of table 1, the solutions of

Pareto frontier optimization are obtained based on the following:
minimum product requirements: SPF of at least 50 and a UVA-PF/SPF protection of at least ⅓ for a specific choice of UV filter combination; and the technical requirement that the determined concentration of the solid UV filters of the Pareto-efficient solutions must be dissolved in the solvents.
optimization objective parameters: minimum filter concentration and minimum solvent concentration.

A linear solubility model comprising the solubilities of each solid UV filters in each solvent; was used to define the technical requirement that the determined concentration of the solid UV filters of the Pareto-efficient solutions must be dissolved in the determined concentration of the solvents of the Pareto-efficient solutions.

TABLE 1
Pareto-efficient solutions

| | | Pareto-efficient | Pareto-efficient | Pareto-efficient |
|---|---|---|---|---|
| | | Solution 1 | Solution 2 | Solution 3 |
| | | Minimized | Minimized | Restriction of |
| | | concentration | concentration | MBBT |
| | | UV filters | solvent | concentration |
| UV filters | Ethylhexyl Triazone | 1.5% | 1.85% | 2.3% |
| | Diethylamino | 0.55% | 2.9% | 3% |
| | Hydroxybenzoyl Hexyl | | | |
| | Benzoate | | | |
| | Bis-Ethylhexyloxyphenol | 2.75% | 0.85% | 1.2% |
| | Methoxyphenyl Triazine | | | |
| | Tris-Biphenyl Triazine | 4.5% | 4.9% | 4.7% |
| | Methylene Bis- | 3.8% | 4.5% | 2.2% |
| | Benzotriazolyl | | | |
| | Tetramethylbutylphenol | | | |
| | Total filter concentration | 13.1% | 15% | 13.4% |
| | | (minimized | | |
| | | concentration | | |
| | | on pareto front) | | |
| Solvents | C12-15 Alkyl Benzoate | 7.5% | 0% | 0% |
| | Dibutyl Adipate | 10.1% | 7.4% | 8.4% |
| | Cocoglycerides | 1% | 0.2% | 1.8% |
| | Dicaprylyl Carbonate | 4% | 0% | 0.4% |
| | Total solvent | 22.6% | 7.6% | 10.6% |
| | concentration | | (minimized | |
| | | | concentration | |
| | | | on pareto front) | |

The optimization signal can be a finite set of points which approximate the Pareto set within a certain accuracy. To support the decision process, it may be preferred to enable a real-time navigation on the continuous Pareto set via the UI of the software application. Therefore, a linear interpolation between the points on the Pareto set may be carried out. This interpolation is not only done in the objective space defined by the set of optimization objective parameters, but also in the design space defined by the substances. The UI of the software application may allow the user to navigate the interpolated solutions by moving sliders corresponding to the optimization objective parameters.

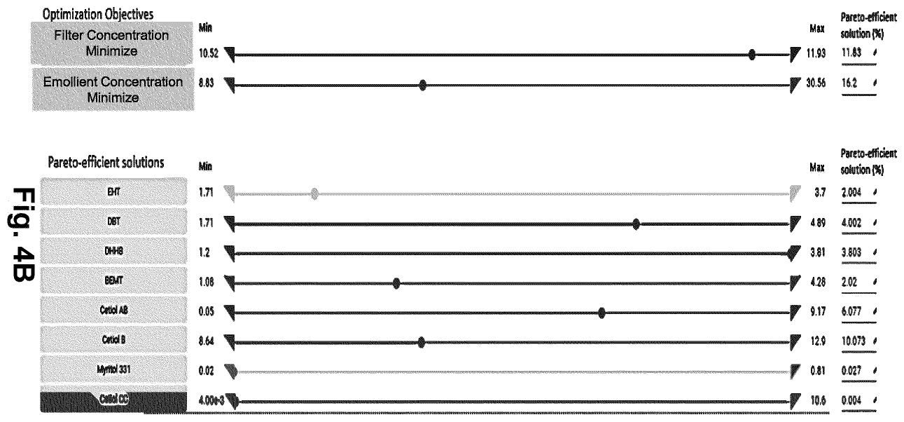

One way the objective sliders work is illustrated in FIG. 5 for the simple case of two optimization objective parameters. During slider change on one objective, a linear problem may be solved such that the remaining optimization objective parameters are all changed in a definite manner. In this way, the complete Pareto set is navigable and it is possible to explore visually the trade-offs between the different optimization objective parameters. This visual exploration can be done in a twofold manner: on the one hand moving one slider causes the displacement of the other sliders according to the shape of the Pareto set. This interaction between the values on the sliders is able to show the best compromises between the conflicting optimization objective parameters. On the other hand, the user can restrict the range of the sliders. Restricting the range of one of the optimization objective parameters, will in general also affect the range of the other optimization objective parameters. This restriction of the decision space is also visualized, yielding information on which alternatives remain feasible and which are now infeasible.

In another example, it may be possible to restrict the range of the sliders of the determined concentration of the composition of the substances such as the concentration of the one or more UV filters (see e.g., solution 3 in table 1).

Although FIGS. 4A and 4B may show an exemplary user interface in form of interactively movable sliders by way of an example, it can be appreciated that other types of graphical control element, such as scrollbar, may be employed for a given implementation.

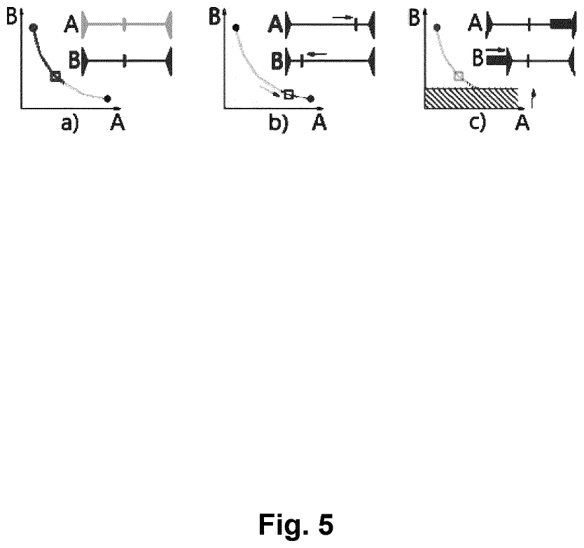

FIG. 6 illustrates a flowchart describing a method 300 for providing manufacturing a sunscreen product.

At block 310, a target performance characteristic of a desired sunscreen product is provided, e.g., via the UI of a software application on an electronic communication device 40 shown FIG. 2. Examples of the target performance characteristic may include, but are not limited to, estimated Sun Protection Factor (SPF), estimated UVA Protection Factor (UVA-PF), critical wavelength, ratio of UVA to UVB protection, ratio of UVA1 to UV protection, blue light protection, Radical protection factor, and environmental impact factor. The user may have the opportunity to select one or more of these properties as sunscreen performance target profile presented on the UI of the software application.

At block 320, based on the target performance characteristic, a composition comprising one or more UV-filter substances and optionally one or more solvents is determined e.g., by the apparatus 10 shown in FIGS. 1 and 2 according to the method described herein. The information about the composition may include identifiers of the substances (e.g., IDs or common or scientific name of the substances) of the determined composition and an amount (e.g., in percentage for finished formulation) of each substance in the composition.

As different formulation types with the same UV-filter composition may exhibit different sun protection performances, the target performance characteristic may be defined for a specific formulation type (e.g., oil in water fluid emulsion, oil in water cream emulsion, etc.). In this example, the composition may be determined based on the target performance characteristic and the specific formulation type such that the difference between the target sun protection performance and the measured sun protection performance in vivo can be reduced. Examples of the formulation type may include, but are not limited to, oil-in-water fluid emulsion, oil-in-water cream emulsion, water-in-oil emulsion, oil-in-water in oil emulsion, water in oil in water emulsion, water in silicone emulsion, silicone-in-water emulsion, polymeric gel cream, lipophilic monophase oil, lipophilic monophase gel, lipophilic monophase stick, lipophilic-alcoholic mixture, hydrophilic monophase fluid, hydrophilic monophase gel, and powder.

In some examples, additional properties characterizing the composition may be provided based on the substances of the composition and the amount of each substance in the composition. Examples of the properties may include, but are not limited to the labelled category SPF, or pass/fail indication of UVA protection for the UVA-PF and critical wavelength according to the definition of the EC recommendation of 22 sept 2006. The information is preferably usable for guiding the production of a sunscreen product.

At block 330, a sunscreen product using the composition comprising one or more of UV-filter substances is manufactured.

At block 340, a measured performance characteristic of the manufactured sunscreen product is provided. For example, the measured performance characteristic may comprise numerous properties, such as achieved performance (SPF and/or UVA-PF), the UVA/SPF ratio, and the total concentration of UV-filter substances.

At block 350, the measured performance characteristic of the manufactured sunscreen product is compared with the target performance characteristic of the sunscreen product to determine if the manufactured sunscreen product fulfils predetermined quality criteria.

The comparison may performed by comparing one or more physical, chemical or physio-chemical characteristic(s) that relate to the performance characteristic. For example, the target SPF may be compared to the measured SPF. The calculated critical wavelength may be compared to the measured critical wavelength of the composition.

The target performance characteristics may be mapped to the measured performance characteristics. In other words, the values corresponding to the performance characteristics may be determined from target performance characteristics. In other embodiments, the measured performance characteristic may be mapped to the target performance characteristics.

Both options are equally applicable.

The target performance characteristics and the measured performance characteristics or any corresponding values derived therefrom are used for validation. Such validation may be performed by comparing values or value ranges.

If the values lie within an acceptable range or value, such as a 1- or 2-standard deviation(s) interval, the produced composition as measured may be valid in the sense that it fulfils the performance criterium or criteria. If the values do not lie within an acceptable range, such as a 1- or 2-standard deviation(s) interval, the produced composition as measured may be invalid in the sense that it does not fulfil the performance criterium or criteria.

If the produced composition is valid, e.g. a control signal for a production process may be triggered at block 360. Such control signal may be associated with the composition of the produced product. It may control dosing equipment for dosing of different substances of the manufactured product in the production process.

If the manufactured product is invalid, e.g. a warning signal for the operator of the production process may be triggered at block 370. Such warning signal may signify the invalidity of the manufactured product. The invalidity may trigger a stop signal for the production process. In such cases, the optimization signal may be updated for the production of the manufactured product to achieve the target performance characteristics of the produced product.

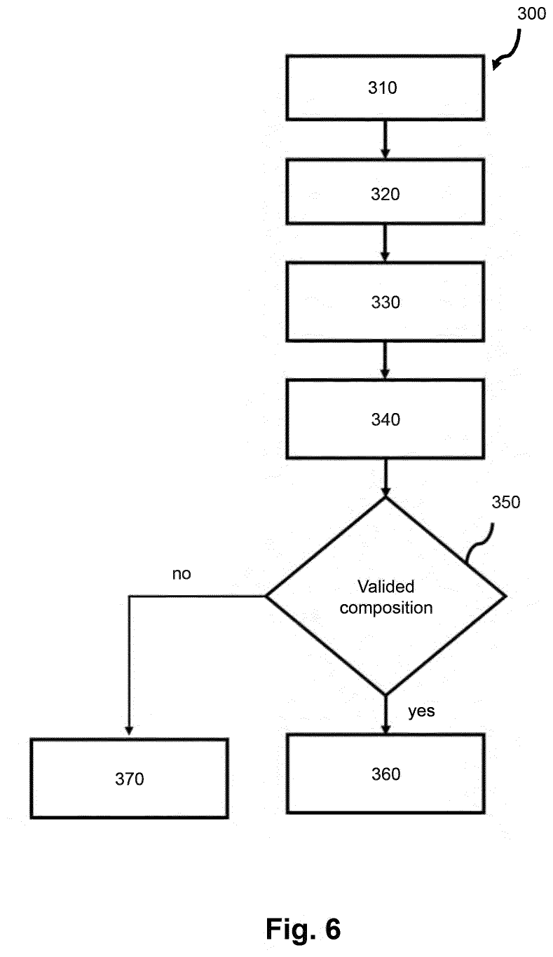

FIG. 7 shows an example of a flowchart describing a method 400 for validating manufacture of a sunscreen product.

At block 410, an existing performance characteristic (e.g. one or more measured physical, chemical, and/or physicochemical properties) of a sunscreen product is provided, which has been produced from one or more substances. The existing performance characteristic may include one or more of SPF, UVA-PF, critical wavelength, ratio of UVA to UVB protection, ratio of UVA1 to UV protection, blue light protection, Radical protection factor, and environmental impact factor.

In some examples, the existing performance characteristic may be provided for a specific formulation type. For example, if the existing sunscreen product is formulated as oil-in-water cream emulsion, the existing performance characteristic may be defined as a performance characteristic for the oil-in-water cream emulsion formulation.

At block 420, based on the existing performance characteristic, a composition is generated according to the method described herein. The generated composition and the existing produced sunscreen product comprise at least one different substance, e.g., for exchanging substances in a sunscreen product. The exchange of a substance can be wished for different reasons such as existence of competitive intellectual property rights, regulatory issues in different countries, or lack of resources.

In some examples, the composition may be determined based on the target performance characteristic and the specific formulation type (e.g., oil-in-water cream emulsion) to reduce the difference between the target sun protection performance and the measured sun protection performance in vivo.

At block 430, based on the generated composition, a product is manufactured.

At block 440, the performance characteristic of the manufactured product and the existing performance characteristics of the sunscreen product are compared to validate the at least one different substance. If the comparison lies within an acceptable range, the at least one new substance is valid. On the other hand, if the comparison does not lie within the acceptable range, the at least one new different substance is invalid.

If the at least one different substance is valid, e.g. control signal is generated for a production process based on the at least one substance may be triggered at block 450. Such control signal may by be associated with the composition of the sunscreen product including the at least one new substance. It may control dosing equipment configured to dose different substances of the sunscreen product in the production process.

If the at least one different substance is invalid, e.g. a warning signal for the operator of the production process may be triggered at block 460. Such warning signal may signify the invalidity of the at least one new substance. This may trigger a stop signal for the production process.

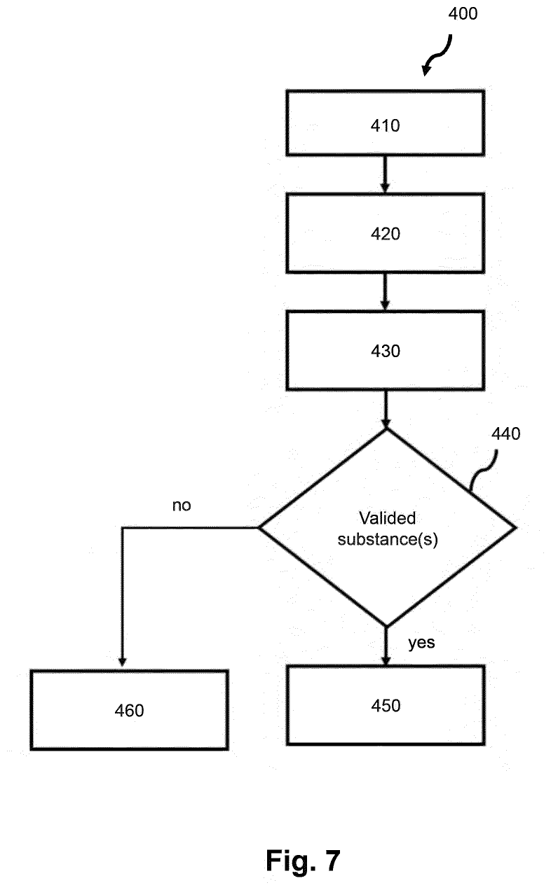

FIG. 8 shows an example of a production line 500 for manufacturing a sunscreen product with a monitoring apparatus 520.

The production line 500 may include dosing equipment 510 configured to dose different substances of the sunscreen product during the production process. The production line 500 may include a conveyor system 530 to convey e.g. bottles, plastic packaging or other suitable packaging to be filled with the sunscreen product. The production line 500 may include a monitoring apparatus 520 configured to monitor quality of the sunscreen product in a production process.

The monitoring apparatus 520 and/or the dosing equipment apparatus 510 may be configured to receive a target performance characteristics of the sunscreen product. The target performance characteristics may specify the composition data for the sunscreen product including one or more UV-filter substances. The target performance characteristics may include quality criteria like SPF. The monitoring apparatus 520 may be configured to provide the composition data to the dosing equipment. The dosing equipment 510 may be configured to control the dosing based on the provided composition data.

The monitoring apparatus 520 may be configured to measure one or more performance characteristics of the produced product. The monitoring apparatus 520 may be configured to compare the physiochemical properties, or any value derived from the physiochemical properties to the measured performance characteristics. If the comparison lies within an acceptable range or value, the produced composition fulfils quality criteria. If the comparison does not lie within an acceptable range or value, the produced composition does not fulfil quality criteria. In the latter case, the monitoring unit may be configured to notify an operator or to provide adjusted composition data to the dosing equipment 510.

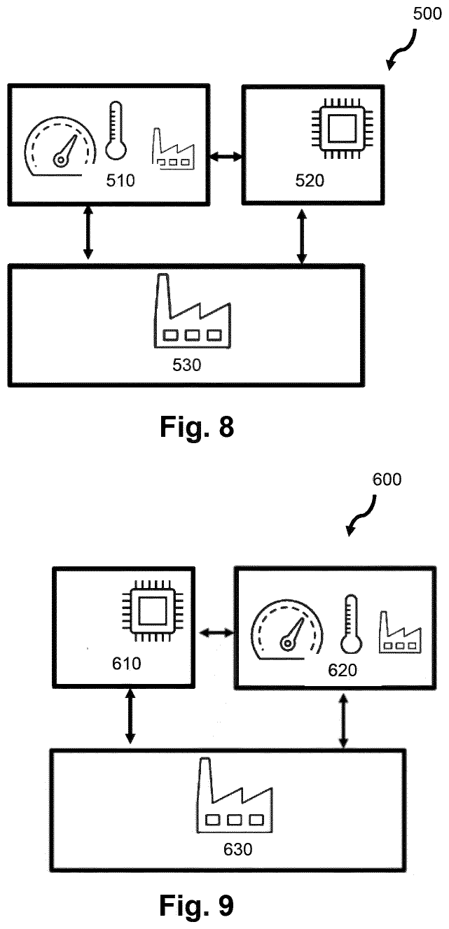

FIG. 9 shows another example of a production line 600 for manufacturing sunscreen product with a validation apparatus 610.

The production line 600 may include dosing equipment 620 configured to dose different substances of the sunscreen product in the production process. The production line 600 may include a conveyor system 630 to convey e.g. bottles, plastic packaging or other suitable packaging to be filled with the sunscreen product. The production line 600 may include a validation apparatus 610 configured to validate the production of the sunscreen product.

The validation apparatus 610 may be configured to receive an existing performance characteristic of the sunscreen product (e.g., SPF, UVA-PF, etc.). The validation apparatus 610 may be configured to generate an optimization signal based on the existing performance characteristic. The optimization signal may comprise information about at least one new substance. The validation apparatus 610 may be configured to validate the at least one new substance for production of the sunscreen product. The validation apparatus 610 may be configured to compare a performance characteristic of a sunscreen product produced using the new optimization signal and the existing performance characteristic. The validation apparatus 610 may be configured to provide the composition data including the at least one new substance to the dosing equipment. The composition data may comprise the amount of the at least one new substance.

Combinations and modifications of the embodiments shown in FIGS. 8 and 9 are similarly possible. Both methods exemplify the strength of the methods as described herein. This allows for simplified and more reliable production through monitoring production of the sunscreen product or through validating new substance(s) to be used for manufacturing the sunscreen product.

In another exemplary embodiment of the present invention, a computer program or a computer program element is provided that is characterized by being adapted to execute the method steps of the method according to one of the preceding embodiments, on an appropriate system. The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfil the procedure of an exemplary embodiment of the method as described above. According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

1.-15. (canceled)

16. A computer-implemented method for determining a composition comprising one or more UV-filter substances, the method comprising the steps of:
a) providing, via an input unit, input data that comprises:
a sunscreen performance target profile comprising a minimum product requirement of the composition; and
a set of optimization objective parameters that comprises two or more optimization objective parameters;
b) performing, by a processing unit, a multi-objective optimizing process on a computational model to determine a set of Pareto-efficient solutions, wherein
each solution is a composition of substances to form the composition such that the composition meets the minimum product requirement; and
each solution is Pareto-efficient with respect to the set of optimization objective parameters; and
c) providing, via an output unit, at least one of the determined composition of substances.

17. The computer-implemented method according to claim 16,
wherein the input data further includes one or more technical requirements to restrict the Pareto-efficient solutions to fulfill the one or more technical requirements.

18. The computer-implemented method according to claim 17,
wherein the one or more technical requirements comprises one or more of:
a solubility model comprising solubility data of each solid filters in one or more solvents;
an assumed limit of individual concentrations of each solvent;
an assumed limit of individual concentrations of each filter;
an assumed limit for a total filter concentration; and
an assumed limit of a total solvent concentration.

19. The computer-implemented method according to claim 16,
wherein each composition of substances comprises at least one of the following:
one or more UV-filter substances;
one or more UV-filter substances and one or more solvents
one or more UV-filter substances in a formulation type; and
one or more UV-filter substances and one or more solvents in a formulation type.

20. The computer-implemented method according to claim 16, further comprising:
providing a graphical user interface, GUI, for Pareto Front visualization, wherein the GUI provides an interactive user interface allowing a user to navigate on the set of determined Pareto-efficient solutions through an adjustment of the set of optimization objective parameters and/or an adjustment of a formulation of the at least one of the determined composition of substances.

21. The computer-implemented method according to claim 20,
wherein an interpolation between points in the set of determined Pareto-efficient solutions is performed, and the GUI provides the interactive user interface allowing the user to navigate on the interpolated Pareto-efficient solutions.

22. The computer-implemented method according to claim 16,
wherein the minimum product requirement of the composition comprises one or more of:
Sun Protection Factor, SPF;
UVA Protection Factor, UVA-PF;
critical wavelength;
ratio of UVA to UVB protection;
ratio UVA1 to UV protection;
blue light protection;
radical protection factor;
limit concentration of a substance
ormulation type, and
environmental impact factor.

23. The computer-implemented method according to claim 16,
wherein the set of optimization objective parameters comprises two or more of:
minimum of required UV-filter concentration;
filtering efficiency;
number of UV-filters used;
type of filters used;
concentration of solvent;
type of solvents used;
minimum concentration of solvent;
highest sun protection factor;
highest UVA protection factor;
highest blue light protection;
ighest environmental friendliness
highest biodegradability,
highest naturality
minimized costs of the composition of substances, and
similarity to a provided composition of filter substances.

24. A method for manufacturing a sunscreen product, the method comprising the steps of:
providing a target performance characteristic of a desired sunscreen product;
determining, based on the target performance characteristic, a composition comprising one or more UV-filter substances according to the method of claim 16; and
manufacturing a sunscreen product using the composition comprising one or more of UV-filter substances.

25. The method according to claim 24, further comprising:
providing a measured performance characteristic of the manufactured sunscreen product; and
comparing the measured performance characteristic of the manufactured sunscreen product with the target performance characteristic of the desired sunscreen product to determine if the manufactured sunscreen product fulfils predetermined quality criteria.

26. A method for validating the manufacturing of a sunscreen product, the method comprising the steps of:
providing an existing performance characteristic for a sunscreen product that has been produced from one or more substances;
generating a composition based on the existing performance characteristic according to the method of claim 16, wherein the generated composition and the existing produced sunscreen product comprise at least one different substance;
producing, based on the generated composition, a product; and
comparing a measured performance characteristic of produced product and the existing performance characteristic of the sunscreen product to validate the at least one substance.

27. An apparatus for determining a composition comprising one or more of UV-filter substances, the apparatus comprising one or more processing units configured to determine a composition of filter substances to form the sunscreen composition, wherein the one or more processing units include instructions, which when executed on the one or more processing units execute the method steps of claim 16.

28. An apparatus for manufacturing a sunscreen product, the apparatus comprising:
a controller module; and
a manufacturing device;
wherein the controller module is configured to control the manufacturing device to manufacture the sunscreen product according to claim 24.

29. An apparatus for validating the manufacture of a sunscreen product, the apparatus comprising one or more processing unit(s) configured to validate production of a sunscreen product, wherein the processing unit(s) include instructions, which when executed on the one or more processing unit(s) preform the method of claim 26.

30. A computer program element comprising instructions, which when executed by a processing unit, cause the processing unit to carry out the steps of the method of claim 16.
